(19) United States
(12) Patent Application Publication
(10) Pub. No.: US 2010/0249247 A1
(43) Pub. Date: Sep. 30, 2010

(54) ANTIMICROBIAL COMPOSITIONS

(76) Inventors: Jeffrey Fallin Andrews, Stillwater, MN (US); William Gerard Cook, Hudson, WI (US)

Correspondence Address: JOEL D. SKINNER, JR.;SKINNER AND ASSOCIATES, 212 COMMERCIAL ST., HUDSON, WI, 54016, US

(21) Appl. No.: 12/414,843

(22) Filed: Mar. 31, 2009

(51) Int. Cl. A61K 31/08; A61P 31/00

(52) U.S. Cl. 514/723

(57) ABSTRACT

An antimicrobial composition for use as a disinfectant or a preservative. The disinfectant may be used in disinfecting and cleaning surfaces. The preservative is particularly useful in personal care and cosmetic products, and pharmaceuticals. The composition includes a base component and an alkyl mono propylene glycol ether, an alkyl mono glycerol ether, or a combination of these ethers. The base component may be water, alcohol, lotion, cream, shampoo, soap, cosmetic color agent, hair conditioner, lip balm, and face, eye or lip make up, ointment.

### 37 C.F.R. §1.71(e) AUTHORIZATION

A portion of the disclosure of this patent document contains material which is subject to copyright protection. The copyright owner has no objection to the facsimile reproduction by anyone of the patent document or the patent disclosure, as it appears in the US Patent and Trademark Office patent file or records, but otherwise reserves all copyright rights whatsoever.

### CROSS-REFERENCE TO RELATED APPLICATIONS, IF ANY

Not applicable.

### STATEMENT REGARDING FEDERALLY SPONSORED RESEARCH OR DEVELOPMENT

Not applicable.

### REFERENCE TO A MICROFICHE APPENDIX, IF ANY

Not applicable.

### BACKGROUND OF THE INVENTION

1. Field of the Invention.

The present invention relates, generally, to antimicrobial compositions and methods. More particularly the invention relates to preservative compositions for use in cosmetic, personal care, and pharmaceutical products; to disinfectant compositions used in residential, commercial, municipal, health care and industrial environments; and to antimicrobial pharmaceutical compositions. Most particularly, the invention relates to the compositions, methods and applications identified above, which contain propylene glycol monoethers, glycerol monoethers, or combinations thereof.

2. Background Information.

The present invention is in the field of antimicrobial agents, disinfectants and preservatives. An antimicrobial is a substance that kills or inhibits the growth of microorganisms (microbes) such as bacteria, fungi, or viruses. Antimicrobial agents either kill microbes (microbicidal) or prevent their growth (microbistatic). Disinfectants are antimicrobial agents that are applied to non-living objects to destroy microorganisms, the process of which is known as disinfection. Preservatives include antimicrobial preservatives, which inhibit the growth of bacteria and fungi. Antimicrobial preservatives are added to other compositions or materials to inhibit such microbe growth in the overall composition or material.

Antimicrobial Agents Used as Disinfectants.

Cleaning products for the cleaning and disinfecting of surfaces, particularly hard or impervious surfaces, commonly use antimicrobial agents. Applications of these cleaners are in the home for bathroom and kitchen cleaning and disinfecting. There are also many industrial uses, especially in food, beverage and meat processing plants. Of course, cleaners with antimicrobial capabilities are used extensively in health care facilities as well. Antimicrobial agents which are or have been on the market in the field of cleaners include:
1 Disinfectant Quaternaries such as Benzalkonium Chloride
2. Phenolic Disinfectants
3. Alkyl (C₈, C₁₀, C₁₂) Monoesters.
4. Hypochlorite bleach
   Entities which are or have been manufacturing such products include Mason, Styson, Lonen, Ecolab, Johnson Wax, Diversey, and Lysol.

The existing compositions in the field of cleaners are believed to have significant limitations and shortcomings. For example, quaternary ammonium compounds are deactivated when formulated with anionic surfactants. Anionic surfactants are powerful detergents and are preferred in many cleaning applications. The ethers of this invention are compatible with all surfactant types; nonionic cationic, amphoteric and anionic, allowing for more flexibility in the formula and better cleaning performance. Antimicrobial effectiveness is reduced however, in the presence of some highly-ethoxylated surfactants such as the Tween® Surfactants (polyoxyetheylene sorbitan surfactants). Quaternary ammonium compounds are also reduced in effectiveness when organic matter is present. The antimicrobial effectiveness of the compositions in this invention is believed to be unaffected by the presence of organic matter.

Hypochlorite bleach is another widely used antimicrobial in cleaning compositions. Hypochlorite is very reactive and reacts with most ingredients typically used in cleaning. Surfactant choice is limited and even when the proper surfactants are used the hypochlorite gradually degrades with time. The level of antimicrobial effectiveness of the new compositions is more stable over time than is the level of antimicrobial effectiveness of hypochlorite disinfectants. Also, hypochlorite disinfectants are irritating to skin, lungs and eyes. Again, the formulator has the entire surfactant palette from which to choose, other than the highly ethoxylated exceptions noted below. Chlorine-containing disinfectants and phenolics are particularly damaging to skin. The propylene glycol monoethers and glycerol monoethers disclosed in this invention are all very mild to the skin.

Antimicrobial Agents Used as Preservatives.

Cosmetic and personal care compositions and pharmaceutical compositions typically use preservatives to control the growth of microbes. Ideally, these preservatives should also be completely non-toxic to humans. Existing classes of preservatives in the field of cosmetic and personal care and pharmaceutical products include:

Standard Preservatives
1. Parabens
2. Dm Dm Hydantoin
3. Isothiazolinone derivatives
4. Kathon
5. Glydant
6. Imidazolidinyl urea

Natural Preservatives
1. Natural extracts that have antimicrobial properties such as garlic, tea tree oil and other plants and plant products.
2. C_{8, 10, 12} monoesters of propylene glycol or glycerine.

The existing preservative compositions in the field of cleaners are believed to have significant limitations and shortcomings. For example, although certain alkyl monoesters have good antimicrobial properties along with mildness to skin, they are susceptible to hydrolysis in the presence of water and therefore are unstable when formulated into a water-based cleaning composition. Other limitations in the prior art include, but are not limited to:
1. Standard preservatives work well at low levels (0.01 to 0.5%) but are potentially harmful.
2. Many natural preservatives work only at relatively high levels in a formula, making them impractical. Typical levels would be 5-10%.
3. C₈, C₁₀, C₁₂ monoesters. Similar to the propylene glycol monoethers and glycerol monoethers in chemical structure and in performance but, because of the ester linkage, hydrolyze in water-based compositions, becoming inactive.
4. Many preservatives used in the market today, for example DM Hydantoin, generate formaldehyde and are potentially harmful.
5. The widely used paraben preservatives are known to be sensitizers in some people.

For these and other reasons, a need clearly exists for the present invention.

All US patents and patent applications, and all other published documents mentioned anywhere in this application are incorporated by reference in their entirety.

### BRIEF SUMMARY OF THE INVENTION

The invention provides propylene glycol (2-hydroxy propyl) monoether and, or glycerol monoether based antimicrobial compositions which may be used as a disinfectant, a preservative or an antimicrobial pharmaceutical. The antimicrobials of the present invention are safe, practical, reliable, and economical, and are believed to fulfill the need and to constitute an improvement over the background technology.

In a primary aspect, this invention provides an antimicrobial composition comprising a base component and an alkyl mono propylene glycol ether and, or an alkyl mono glycerol ether wherein the base component is selected from the group consisting of water, alcohols, lotion, cream, shampoo, soap, cosmetic color agent, hair conditioner, lip balm, and face, eye or lip make up, ointment or the like.

The aspects, features, advantages, benefits and objects of the invention will become clear to those skilled in the art by reference to the following description and claims.

### DETAILED DESCRIPTION

The present invention comprises compositions which utilize certain propylene glycol (2-hydroxy propyl) monoethers, certain glycerol monoethers, or combinations thereof, as antimicrobial agents and which have distinct advantages over materials currently in use. These new antimicrobial compounds have the following basic structure:

For Propylene glycol (2-hydroxy propyl) monoethers:

R—O—CH₂—CHOH—CH₃

Where R is either C₁₂H₂₅, C₁₀H₂₁ or C₈H₁₇

For Glycerol monoethers:

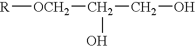

Where R is either C₁₂H₂₅, C₁₀H₂₁ or C₈H₁₇

Propylene glycol monoethers and glycerol monoethers are completely stable to hydrolysis and therefore are an improvement over antimicrobials, including antimicrobials containing an ester linkage.

The monoethers are active against Gram positive bacteria, fungi, yeasts, and lipid coated viruses. When formulated with a proper synergist they are also active against Gram negative bacteria making them truly broad spectrum antimicrobial agents. Effective synergists include commonly used cosmetic and personal care product components such as disodium EDTA, tetrasodium EDTA, lactic acid and citric acid. Other effective synergists include mandelic acid, tartaric acid, adipic acid, acetic acid, succinic acid, ascorbic acid, benzoic acid, salicylic acid, ethanol, isoproanol, octanol, acidic sodium, hexametaphosphate, sodium, acid pyrophosphate and decanol. It should be noted that salts of the listed acids are not active as synergists for the subject monoethers with the exception of the listed EDTA salts and phosphate salts.

Antimicrobial Composition For Use as Disinfectant.

The basic composition of the present invention with respect to an antimicrobial disinfectant solution is water and an alkyl mono propylene glycol (2-hydroxy propyl) ether and, or an alkyl mono glycerol ether. An alkyl group is C₈, C₁₀, C₁₂. The concentration of alkyl mono propylene glycol ethers and glycerol monoethers is at least 0.01%, and preferably between 0.10 and 10 percent (%) by weight. The most preferred range is 0.1 to 1.0% by weight.

The composition may include one or more of the following further constituents or components: a surfactant, a synergist, a chelant, inorganic acids, organic acids or bases or salts. Synergists may include one or more of the following: mandelic acid, tartaric acid, adipic acid, acetic acid, succinic acid, ascorbic acid, benzoic acid, salicylic acid, ethanol, isoproanol, octanol, decanol, disodium EDTA, phosphate salts, tetrasodium EDTA, lactic acid, and citric acid. Blends may further include other cleaning agents to make a disinfectant/cleaner. It is also within the purview of the invention that it could be used as a spray to eliminate odors.

So a preferred embodiment of an antimicrobial disinfectant cleaner composition comprises a surfactant, a synergist, water, and an inorganic acid, organic acid, base or salt in combination with one or more alkyl mono propylene glycol ethers and/or one or more alkyl mono glycerol ethers.

### EXAMPLE 1

A disinfectant for hard surfaces includes:
Propylene Glycol C12 (PGC12) monoether-1.0% by weight
Dioctyl sodium sulfosuccinate-2.0
Pluronic P65-10.0
Disodium EDTA-0.10
Isopropanol-15.0
Deionized water-71.90
   In this example, the PGC12 monoether is present as an active antimicrobial agent used to disinfect hard surfaces such as counter tops, sinks, shower stalls and the like in homes, commercial establishments, heath care facilities (such as nursing homes and hospitals), restaurants. Disodium EDTA is present as a synergist to enhance monoether activity against Gram negative bacteria. In this and all other examples, all percentages of ingredients are percent by total weight of the formulation.

### EXAMPLE 2

A shower and bath disinfectant cleaner includes:
Glycerol C10 monoether-2.0% by weight
Mackamide C10-1.50
Tetrasodium EDTA-1.0
Dioctyl sodium sulfosuccinate-3.0
Isopropanol-15.0
Deionized water-77.50
   In this example, the monoether is used as an active antifungal—antimildew agent in a typical shower and bathroom wall cleaner.

In general, the composition of the invention is mild, non toxic, and effective against microbes. The composition kills bacteria, mold, fungi and lipid coated viruses on hard surfaces, and depending on composition can also clean the surface of dirt and oils. The ethers will also help emulsification in some formulas.

The composition is an improvement over Quaternary Ammonium Compounds insofar as being (1) more flexible in that they can be formulated with anionic surfactants; “quats” cannot; and (2) more effective when there is a heavy organic load on the surface to be cleaned. The composition is an improvement over hypochlorite bleach insofar as being (1) milder; (2) more compatible with other cleaning ingredients; and (3) less hazardous. The composition is an improvement over alkyl esters insofar as being stable to hydrolysis. And, the composition is an improvement over phenolics insofar as being much less toxic.

Antimicrobial Composition for Use as Preservative in Personal Care and Cosmetic Products, and in Pharmaceuticals.

The composition of the invention is especially suitable as a preservative for use with or incorporation into any cosmetic, personal care or pharmaceutical composition which is fully or partially preserved (has inhibited microbial growth) with the subject ethers, including, generally:
1. Lotion
2. Cream
3. Shampoo
4. Liquid hand soap
5. Color Cosmetic
6. Hair Conditioner
7. Body Wash
8. Make up
9. Lip Product
10. Ointment
11. Skin Moisturizer

So, the basic composition of the present invention comprises one of the above identified personal care or cosmetic or pharmaceutical compositions and an alkyl mono propylene glycol ether or glycerol monoether. An alkyl group is C₈, C₁₀, C₁₂. The concentration of alkyl mono propylene glycol ethers, or of alkyl glycerol monoethers is at least 0.01% by weight, and preferably between 0.10 and 10% by weight. The most preferred range is 0.1 to 1.0% by weight.

The composition may include a synergist, water or inorganic acid, organic acid, base or salt. Synergists which may be used in cosmetic and personal care and pharmaceutical products include but are not limited to chelating agents such as disodium EDTA, tetrasodium EDTA, lactic acid and citric acid. The composition is non-toxic and will not support microbial growth to the extent that the products become outside of accepted guidelines and standards, unsafe or unusable. The composition could accurately claim to be “free of harmful preservatives” depending upon the applicable law and regulations in the distribution area.

### EXAMPLE 3

A shampoo formulation includes:
PGC 8 monoether-0.10% by weight
PCG 10 monoether-0.10
Benzoic acid-0.10
Sodium lauryl ether sulfate-40.0
Mackamide C-2.0
Mackamide WGB-5.0
Deionized water-52.70
   In this example, two (2) monoethers are use as antimicrobial preservatives in a typical shampoo formulation. Benzoic acid is used as a synergist to enhance monoether activity against Gram negative bacteria.

### EXAMPLE 4

A moisturizing non-aqueous ointment formulation includes:
Glycerol C12 monoether-0.25% by weight
Glycerol C10 monoether-0.25
Glycerol C8 monoether-0.25
Benzoic Acid-0.10
Lanolin USP-10.0
White bees wax-5.0
Fionsolv TN-5.0
White petroleum USP-79.15
   In this example, a combination of three (3) monoethers is used to preserve a typical moisturizing ointment base that can be used as a skin moisturizer or as a pharmaceutical ointment base to carry active drug agents.

### EXAMPLE 5

A liquid moisturizing skin disinfectant drug formulation includes:
PGC 12 monoether-1.0% by weight
PGC 8 monoether-1.0
Lactic acid-1.0
Ethanol-97.00
   This example shows the use of a combination of propylene glycol C8 monoether and the PGC 12 monoether with lactic acid as a synergist dissolved in ethanol to make a liquid moisturizing skin disinfectant drug product. The ethanol alone is an excellent skin antiseptic but has the drawback of being extremely drying to the skin. The monoethers add two advantages over plain ethanol. Along with the lactic acid synergist they help prevent drying of the skin and they leave a long lasting antimicrobial film on the skin. In contrast, plain ethanol alone evaporates quickly from the skin and therefore has no lasting effect.

The process to make the cosmetics, personal care or pharmaceutical products, incorporating the preservative composition is within standard practice in the formulation industry. The composition may be made for example in a blending unit.

Antimicrobial Composition For Use as a Pharmaceutical.

The composition of the invention may be used as a pharmaceutical agent, where the antimicrobial function is used to treat disease directly. The basic composition of the present invention with respect to an antimicrobial pharmaceutical is water and an alkyl mono propylene glycol (2-hydroxy propyl) ether and, or an alkyl mono glycerol ether. An alkyl group is C₈, C₁₀, C₁₂. The concentration of alkyl mono propylene glycol ethers and glycerol monoethers is at least 0.01%, and preferably between 0.10 and 10 percent (%) by weight. The most preferred range is 0.1 to 1.0% by weight. The pharmaceutical composition may include a synergist, water or inorganic acid, organic acid, base or salt.

The descriptions above and the accompanying materials should be interpreted in the illustrative and not the limited sense. While the invention has been disclosed in connection with the preferred embodiment or embodiments thereof, it should be understood that there may be other embodiments which fall within the scope of the invention.

The invention claimed is:

1. An antimicrobial composition comprising a base component and an alkyl monoether selected from the group consisting of an alkyl mono propylene glycol (2-hydroxy propyl) ether, and an alkyl mono glycerol ether.

2. The composition of claim 1, wherein the alkyl monoether is the C₈, C₁₀, or C₁₂ alkyl mono propylene glycol ether.

3. The composition of claim 1, wherein the alkyl monoether is the C₈, C₁₀, or C₁₂ alkyl mono glycerol ether.

4. The composition of claim 1, wherein the base component is water.

5. The composition of claim 1, wherein the base component is an alcohol.

6. The composition of claim 1 wherein the base component is selected from the group consisting of a lotion, a cream, shampoo, liquid hand soap, color cosmetic, hair conditioner, body wash, make up, lip product, ointment and skin moisturizer.

7. The composition of claim 1, wherein the base component is selected from the group of components consisting of lotion, cream, shampoo, soap, ointment, cosmetic color agent, hair conditioner, lip balm, and face, eye or lip make up, or skin moisturizer.

8. The composition of claim 1, wherein the alkyl mono propylene glycol ether or alkyl glycerol monoether is provided in an amount of at least 0.01 % by weight.

9. The composition of claim 8, wherein the alkyl mono propylene glycol ether or alkyl mono glycerol ether is provided in an amount between 0.1 and 10% by weight.

10. The composition of claim 9, wherein the ether is provided in a range of 0.1-1.0% by weight.

11. The composition of claim 1, further comprising a surfactant.

12. The composition of claim 1, further comprising a chelant.

13. The composition of claim 1, further comprising at least one organic and inorganic acid, base or salt.

14. The composition of claim 1, further comprising a cleaner.

15. The composition of claim 1, further comprising a synergist.

16. The composition of claim 10, wherein the synergist is selected from the group consisting of malic acid, mandelic acid, tartaric acid, adipic acid, acetic acid, succinic acid, ascorbic acid, benzoic acid, salicylic acid, ethanol, isoproanol, octanol, decanol, disodium EDTA, tetrasodium EDTA, lactic acid, citric acid, sodium acid, pyrophosphate, acidic sodium hexametaphosphate.

17. The composition of claim 1, further comprising a fragrance.

18. The composition of claim 1, further comprising at least one pharmaceutical constituent.

19. The composition of claim 1, wherein the composition is used as a disinfectant cleaner.

20. The composition of claim 1, wherein the composition is used as a preservative.

21. The composition of claim 20, wherein the preservative is used to preserve a personal care or cosmetic product, or a pharmaceutical product.

22. The composition of claim 1, used as an active antimicrobial drug in a pharmaceutical formulation to kill bacteria, fungi and lipid coated virus disease causing organisms.

23. A disinfecting composition comprising:
a. an alkyl mono propylene glycol ether, an alkyl mono glycerol ether or a combination of the two, in a predetermined amount by weight;
b. a base component, and
c. a synergist.

24. The disinfecting composition of claim 23, wherein the alkyl mono propylene glycol ether, alkyl mono glycerol ether, or combination thereof, is provided in an amount of at least 0.01% by weight.

25. A preservative composition comprising:
a. an alkyl mono propylene glycol ether, an alkyl mono glycerol ether or a combination of the two, in a predetermined amount by weight; and
b. a base component selected from the group of components consisting of water, lotion, an alcohol, cream, shampoo, soap, a cosmetic color agent, a cosmetic, moisturizer, ointment, conditioner, lip balm, and face, eye or lip make up.

26. The composition of claim 25, wherein the alkyl mono propylene glycol ether, an alkyl mono glycerol ether or a combination thereof, is provided in an amount of at least 0.01% by weight.
